(19) United States
(12) Patent Application Publication
(10) Pub. No.: US 2008/0015539 A1
(43) Pub. Date: Jan. 17, 2008

(54) Bottle with adapter for receiving needleless syringe

(76) Inventors: Robert Pieroni, Lewes, DE (US); Curt Metzbower, Washington Crossing, PA (US)

Correspondence Address: DANIEL W. SULLIVAN;DENTSPLY INTERNATIONAL INC., 570 WEST COLLEGE AVENUE, P.O. BOX 872, YORK, PA, 17405-0872, US

(21) Appl. No.: 11/364,780

(22) Filed: Feb. 28, 2006

(51) Int. Cl. A61J 3/00; A61B 19/00

(52) U.S. Cl. 604/403

(57) ABSTRACT

An adapter assembly for connecting a medication bottle to a needleless syringe, particularly a luer-lock syringe, is provided. The bottle adapter includes a cylindrically-shaped main body. Multiple annular fins project outwardly from the main body for engaging and gripping the interior surfaces of the bottle. This close interference fitting between the adapter and bottle provides a tight and effective seal. The adapter is particularly suitable for connecting a bottle containing a drug in solid form, such as a powdered drug, to a syringe containing a liquid carrier. The syringe is inserted into the adapter and liquid is injected into the bottle. The liquid mixes with the powdered drug to form a drug solution. The bottle adapter can be used in a wide variety of medical, dental, pharmaceutical, and other healthcare applications.

### BACKGROUND OF THE INVENTION

1. Field of the Invention

The present invention relates generally to an adapter for connecting a medication bottle to a needleless syringe, particularly a luer-lock syringe. The adapter is particularly suitable for connecting a bottle containing a drug in solid form, such as a powdered drug, to a syringe containing a liquid. A clinician inserts the syringe into the adapter and depresses the syringe plunger to eject the liquid into the bottle. The liquid mixes with the powdered drug and the resulting drug solution can be administered to a patient.

2. Brief Description of the Related Art

There are many known methods for mixing a powdered drug with a liquid carrier. Many of these methods involve using a needle, spike, or other penetrating device. Such methods involve storing the drug in a glass vial that is sealed with a rubber or plastic membrane. The needle is used to pierce the membrane and deliver the liquid carrier into the vial.

For example, Thibault et al., U.S. Pat. No. 5,873,872 discloses a connector assembly that can be installed on a vial, such as a vial containing a lyophilized drug, so that liquid can flow efficiently into and/or out of a vial. The connector assembly includes a spike slidably mounted in the open top of the vial. The connector assembly further includes a stopper sealingly engaged in the open top of the vial. The connector assembly slides in response to the axially movement of the spike. Movement of the spike relative to the vial causes the stopper to move in and out of sealing engagement with the vial.

In other instances, there may be a need to withdraw liquid from a medicine vial, and needless syringes can be used in such procedures. For example, Smith, U.S. Pat. Nos. 4,230,112 and 4,317,448 disclose a nozzle for connecting a liquid dispensing syringe with the interior of a bottle containing liquid medication. The nozzle includes a tubular body member made of a resilient material that fits snugly within the mouth of the bottle. Projections or beads are provided on the tubular body for engaging the inside surface of the bottle. An opening extends axially through the body for receiving the nozzle of the syringe. The opening includes a forward nozzle-receiving portion and a rear portion. The forward portion is tapered to receive the nozzle of the syringe and provide a tight fit so that fluid does not flow around the nozzle. The rear portion of the opening is enlarged and has a conical shape.

Shimp et al., U.S. Pat. No. 4,784,657 is directed to a method for transferring solid granule materials from a vial to a syringe. The vial is fitted with an “inter-connector device,” which features a tapered sleeve for receiving one end of the syringe. The seal between the tapered sleeve and syringe prevents the granules from spilling. The granules may be transferred with one hand by inverting the vial/syringe assembly. The granules may be re-transferred to the vial by simply turning the assembly right side-up again without spilling. A metering port located between the tapered sleeve and interior of the vial prevents the granules from coming to rest in the sleeve.

Watson et al., U.S. Pat. No. 5,598,939 discloses a syringe fitting positioned within the cap of a medicine bottle. The fitting is positioned within the fluid passageway of the cap and is configured for receiving the hub of a needleless syringe. A flexible, plastic tube having one end in fluid communication with the fitting and an opposing end near the bottom of the bottle is placed within the bottle. The syringe can be inserted into the fitting and used to draw liquid from the bottle. The liquid flows through the tube and into the syringe.

Brony, U.S. Pat. No. 5,620,434 discloses a link assembly connecting a needleless syringe and a liquid medicine vial. An adapter flange in the link forms both an anchor for a conventional luer-lock syringe and a receiving receptacle for a conventional slip-tip syringe. The bottom end of the link assembly is capped with a diaphragm-like portion containing a series of tiny holes. The holes form a sieve and serve to provide multiple flow paths for the fluid. The tiny holes also provide some filtering of the fluid. A valve automatically seals the vial to protect against loss of fluid and contamination in the event that the vial is overturned.

As discussed above, there are known adapter or connector systems that can be used with needless syringes to withdraw liquid or solid granules from a medicine vial or bottle. Still, there is a need for an adapter that can be used to connect a bottle containing a drug in solid form, such as a powdered drug, to a syringe containing a liquid. Such an adapter is needed, because it could be used to interconnect the bottle with the syringe so that the liquid could be transferred from the syringe to the bottle and the resulting drug solution could be withdrawn from bottle using the same syringe. More particularly, such an adapter assembly could be used to secure a syringe containing a liquid carrier for a powdered drug. Depressing the plunger of the syringe would cause the carrier to be injected into the bottle, thereby dissolving the powdered drug. Then, the same syringe could be used to withdraw the drug solution from the bottle. The adapter should be constructed so as to allow liquid to be transferred between the syringe and bottle in such a manner that there is no substantial leaking or spilling of the fluid. The present invention provides such an adapter assembly having these desirable features as well as other advantageous characteristics and properties.

### SUMMARY OF THE INVENTION

The present invention relates to an adapter assembly for connecting a medication bottle to a needleless syringe, particularly a luer-lock syringe. The adapter is particularly suitable for connecting a bottle containing a drug in solid form, such as a powdered drug, to a syringe containing a liquid carrier. The syringe is inserted into the adapter and the liquid is injected into the bottle. The liquid mixes with the drug to form a drug solution.

The adapter includes a cylindrically-shaped main body member with an upper annular rim that defines an upper recessed portion. A tubular member, having a through-bore extending in an axial direction through the body member, is disposed within the recessed portion. The tubular member includes an upper portion for receiving the tip of the luer-lock syringe and a lower portion that provides a fluid passageway to the interior of the bottle. The upper portion of the tubular member preferably has a slightly tapered cylindrical structure with a relatively small inside diameter. The lower portion of the tubular member preferably has a conical-shaped structure with a relatively large inside diameter. The adapter includes multiple flexible annular fins that project outwardly from the main body and grip the interior surfaces of the bottle. Upper and lower annular fins are included in the structure. Intermediate fins can be disposed between the upper and lower fins. Because of these radiating annular fins, there is a close interference fit between the adapter and bottle. In effect, the adapter seals the bottle. The adapter further includes an upper annular rim that extends outwardly and overlays the upper rim of the bottle. This mating of the upper rims further helps create a tight seal between the adapter and bottle. The lower portion of the tubular member is funnel-shaped and contains a lower annular face. It is preferred that the lower face be substantially flush with the lower annular fin that radiates outwardly from the adapter. As described further below, this structure provides a better funneling effect and allows the drug solution to be more effectively drawn from the bottle.

In another embodiment, the adapter further includes a lower flange extending from the lower portion of the tubular member. Here, the lower face of the tubular member is not flush with the lower annular rib radiating from the adapter body. The lower flange includes a drainage notch for providing a liquid passageway between the tubular member and interior of the bottle. In still another embodiment, the adapter includes a conduit that extends in an axial direction and is parallel to the tubular member. The conduit includes top and bottom openings for venting air pressure from the interior of the bottle.

### BRIEF DESCRIPTION OF THE DRAWINGS

The novel features that are characteristic of the present invention are set forth in the appended claims. However, the preferred embodiments of the invention, together with further objects and attendant advantages, are best understood by reference to the following detailed description in connection with the accompanying drawings in which:

FIG. 1 is a cross-sectional view of one embodiment of the adapter assembly of the present invention showing the adapter positioned in a medication bottle and a luer-lock syringe locked in the adapter;

FIG. 1A is an exploded perspective view of the medication bottle in FIG. 1 with the adapter assembly and luer-lock syringe removed;

FIG. 2 is a side perspective view of one embodiment of the adapter assembly of the present invention;

FIG. 2A is a top perspective view of the adapter assembly shown in FIG. 2;

FIG. 3 is a side perspective view of one embodiment of the adapter assembly of the present invention;

FIG. 3A is a cross-sectional view along Line A-A of FIG. 3;

FIG. 4 is a cross-sectional view of one embodiment of the adapter assembly of the present invention, wherein the lower face of the tubular member is flush with the lower annular rib;

FIG. 5 is a cross-sectional view of one embodiment of the adapter assembly of the present invention, wherein the lower face of the tubular member is not flush with the lower annular rib;

FIG. 6 is a top perspective view of one embodiment of the adapter assembly of the present invention showing a vent in the adapter for relieving air pressure;

FIG. 6A is a cross-sectional view along Line A-A of FIG. 6;

FIG. 7 is a cross-sectional view of one embodiment of the adapter assembly of the present invention showing the adapter positioned in a medication bottle; and

FIG. 8 is a perspective view of one embodiment of the adapter assembly of the present invention showing the adapter positioned in a medication bottle and a luer-lock syringe being inserted into the adapter.

### DETAILED DESCRIPTION OF THE PREFERRED EMBODIMENTS

The present invention relates to an improved adapter assembly for connecting a medication bottle to a needleless syringe, particularly a luer-lock syringe. By the term, “luer-lock syringe,” it is meant any type of syringe device having a luer-lock adapter and not having a needle. Luer-lock syringes and their mechanisms of operation are well known in the art. By the term, “bottle,” it is meant any type of rigid or semi-rigid container including, but not limited to, vials, flasks, cartons, and other devices. The bottle may be made of glass, plastic, or any other suitable material.

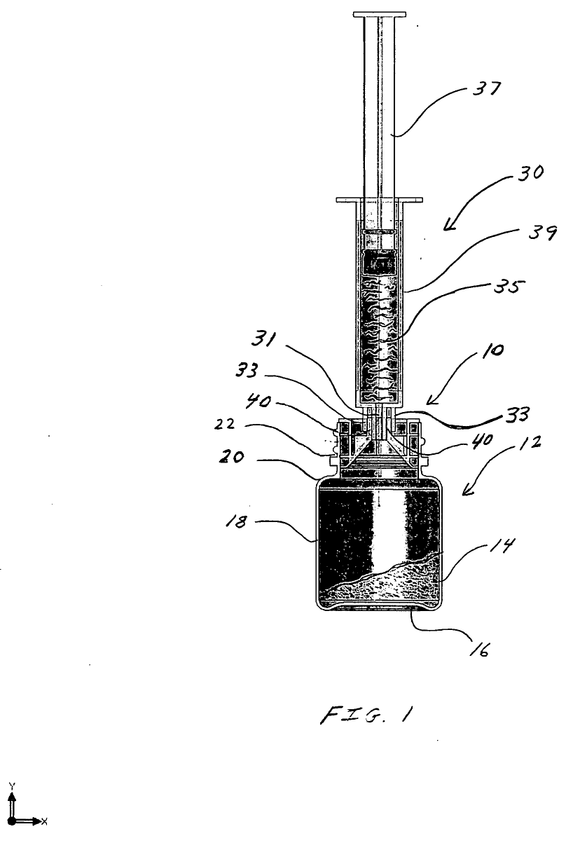

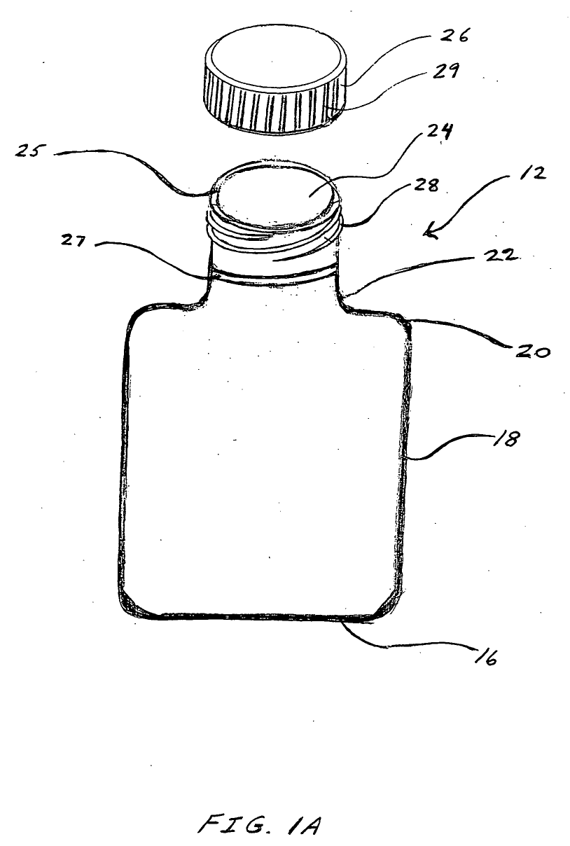

Referring to the drawings, FIG. 1 shows one embodiment of the adapter assembly of the present invention generally designated at (10). In FIG. 1, the adapter assembly (10) is shown attached to a cylindrical medicine bottle (12) containing a powdered drug (14). A luer-lock syringe (30) containing a liquid (35) is shown inserted and locked in the adapter (10). The bottle (12) includes a bottom wall (16) and a cylindrical side wall (18) extending upwardly from the bottom wall (16). A shoulder portion (20) extends inwardly and upwardly from the top portion of the cylindrical side wall (18) to a neck portion (22). Extending upwardly from the shoulder portion (20), the neck (22) defines a top opening (24) (not shown in FIG. 1). The adapter (10) and inter-locking of the luer-lock syringe (30) therein are described in further detail below.

Turning to FIG. 1A, the bottle (12) is shown with the adapter assembly (10) removed and the top opening (24) clearly visible. An upper annular rim (25) surrounds the top opening (24). In addition, a lower annular rib (27) is located in the lower region of the neck portion (22). When a clinician is ready to insert the syringe (30) into the adapter (10), he or she can grip the lower rib (27) with one hand and insert the syringe (30) with the other hand. The lower rib (27) can serve other functions as well. For example, a tamper-resistant plastic shrink band (not shown) can be placed over the cap (26). Such plastic shrink bands are well known in the art. The plastic band is wrapped over the cap (26) and lower rib (27) to provide added protection and safety. As the cap (26) is twisted off, the plastic band breaks. When the plastic band is in a non-broken condition, it provides a seal. This helps protect against possible tainting and tampering of the powdered drug (14) contained within the bottle (12). Foreign debris is less likely to be introduced into the bottle (12) and contamination risks are minimized. Furthermore, the plastic seal helps make the bottle more “child-safe.”

In FIG. 1A, the outer surface of the neck portion (22) is shown as being threaded (28). A cap (26) with a concentric internal thread for engaging the outer thread (28) of the neck portion (22) is included with the medication bottle (12). The cap (26) is used to cover the opening (24) of the bottle (12). The cap (26) can be placed over the opening (24) with the adapter (10) positioned therein. In other words, the adapter (10) does not interfere with placing the cap (26) on or taking the cap (26) off the bottle (12). As discussed further below, it can be advantageous to place the adapter (10) within the bottle (12) prior to shipping the bottle (12) to a clinician. In such a system, when the clinician is ready to prepare a drug solution, he or she easily unscrews the cap (26). The opened bottle (12) contains the adapter (10) already in place. More particularly, in one example, the clinician can hold the body of the bottle (12) with his or her right hand. Then, he or she can grip the cap (26) between the thumb and finger of his or her left hand. To make the cap (26) easier to grip, the cap can include ribs (29) on its outer surface. It should be understood that a “screw-on” cap (26), which engages a threaded outer surface (28) of the neck (22), is only one of the many possible closure systems that can be used for the medication bottle (12). The closure system comprising a threaded cap (26) and threaded surface (28) is shown for illustration purposes only and should not be considered restrictive. Other closure systems such as, for example, snap-fitting caps may be used in accordance with this invention.

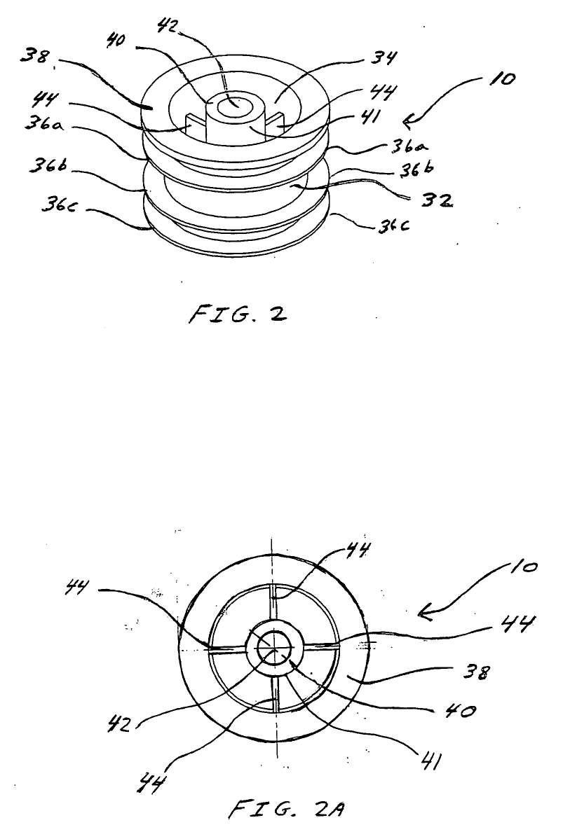

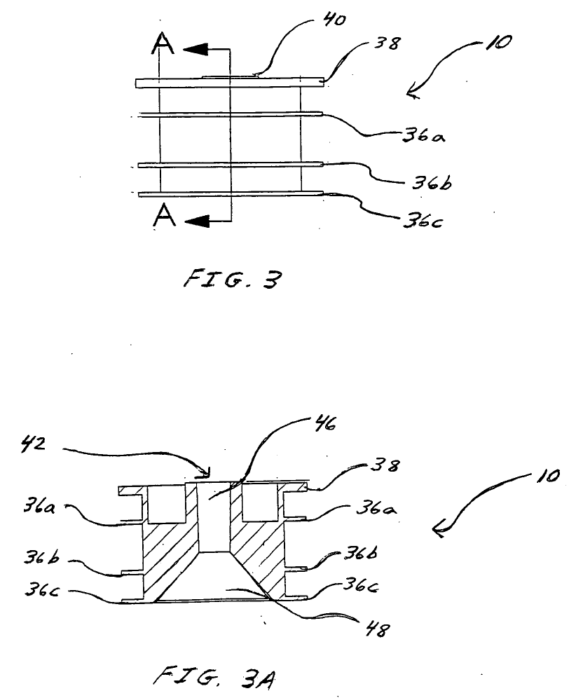

Referring to FIG. 2, one embodiment of the adapter assembly (10) is shown. The adapter (10) includes a cylindrically-shaped main body (32) with an upper recessed portion (34). Multiple annular fins (36a, 36b, 36c) project outwardly from the main body (32). In the structure shown in FIG. 2, the annular fin (36a) is an upper fin and annular fin (36c) is a lower fin, while the annular fin (36b) is an intermediate fin. The adapter (10) includes at least one upper and lower annular fin (36a, 36c). The intermediate annular fin (36b) is optional. One or more intermediate fins (36b) can be interposed between the upper and lower annular fins (36a, 36c) if desired. The intermediate fin (36b) helps improve stability and the interference fit between the adapter (10) and interior surfaces of the bottle (12). The adapter (10) is shown having three annular fins (36a, 36b, 36c) for illustration purposes only and this number should not be considered restrictive. The adapter (10) may include any appropriate number of radiating fins (36a, 36b, 36c). This will vary based upon the geometry and dimensions of the adapter (10). The adapter (10) further includes an upper annular rim (38). When the adapter (10) is positioned in the medication bottle (12), the annular fins (36a, 36b, 36c) engage and grip the interior surfaces of the neck portion (22), while the upper rim (38) of the adapter (10) fits over the upper rim (25) of the bottle (12). As the annular fins (36a, 36b, 36c) grip the interior surfaces of the neck (22) and the upper rim (38) overlays the rim (25), the opening (24) of the bottle (12) is effectively sealed.

As also shown in FIG. 2, the recessed portion (34) of the adapter (10) includes a tubular member (40) extending upwardly. The tubular member (40) contains an axial through-bore (42), which is adapted for receiving the tip (31) of the luer-lock syringe (30). The diameter of the through-bore (42) is appropriately sized to tightly secure and seal the syringe tip (31) in place. The step of inserting and locking the luer-lock syringe (30) in the through-bore (42) is described in further detail below. The adapter (10) further includes support ribs (44), which extend outwardly from the upstanding tubular member (40) and are fastened to the interior surfaces of the adapter (10). Preferably, the adapter (10) contains four support ribs (44). FIG. 2 is a side perspective view of the adapter (10) and only two of the four support ribs (44) are shown in this view. In FIG. 2A, a top perspective view of the adapter (10) is shown, and the four support ribs (44) are clearly visible in this view. The number of support ribs (44) shown in FIGS. 2 and 2A is for illustrative purposes only and should not be considered restrictive.

The adapter (10) is a unitary, single-piece structure and the annular fins (36a, 36b, and 36c), tubular member (40), and support ribs (44) are integrally molded components. The adapter (10) can be made from any suitable polymeric material including, but not limited to, acetals, acrylics, polyamides, polyesters, polycarbonates, polyolefins, polystyrene, and polyvinyl chloride. For example, the adapter (10) can be molded from high-density polyethylene (HDPE) or low-density polyethylene (LDPE). The annular fins (36a, 36b, and 36c) should be somewhat flexible so that the adapter (10) can be inserted more easily into the neck portion (22) of the bottle (12). As the adapter (10) is being inserted, the resilient annular fins (36a, 36b, and 36c) tend to flex slightly upwards. After the adapter (10) has been positioned within the neck (22), the annular fins (36a, 36b, and 36c) resume their natural shape and flex outwardly so as to engage the interior surfaces of the neck (22).

Referring to FIGS. 3 and 3A, the through-bore (42) includes an upper portion (46) having a cylindrically-shaped structure and a lower portion (48) having an inverted cone structure. The inside diameter of the upper portion (46) of the through-bore (42) is relatively small. In addition, the upper portion (46) is slightly tapered, allowing the luer-lock syringe tip (31) to be fitted tightly therein. As an example, the top region of the tapered upper portion (46) can have an inside diameter in the range of about 0.165 to about 0.160 inches, while the bottom region of the upper portion (46) can have a diameter in the range of about 0.160 to about 0.155 inches. The outside diameter of the upper portion (46) can be, for example, in the range of about 0.285 to about 0.310 inches. By contrast, the small inside diameter of the lower funnel portion (48) is relatively large. As an example, the small inside diameter of the funnel (48) can be in the range of about 0.160 to about 0.155 inches. Meanwhile, the large inside diameter of the funnel (48) can be, for example, in the range of about 0.610 to about 0.810 inches. The upper portion (46) of the through-bore (42) provides a guide for inserting the syringe tip (31) therein. In the luer-lock syringe (30), a small cylindrical housing (33) having a threaded interior (not shown) surrounds the tip (31). A clinician inserts the syringe tip (31) into the through-bore (42) of the adapter (10) and locks the syringe (30) in place by pressing downwardly on the syringe (30) and twisting it. Normally, the syringe (30) is twisted in a clockwise direction to lock it in the adapter (10). Referring to FIG. 1, as the syringe (30) is twisted, the threaded interior of the housing (33), surrounding the syringe tip (31), grips and bites the smooth outer surface (41) of the tubular member (40). Because a relatively soft plastic material is used to make the tubular member (40), the threaded interior of the housing (33) is able to bite into the smooth outer surface (41). (See also FIG. 2.) The syringe (30) is locked effectively in place as the threaded housing (33) engages the tubular member (40). The syringe tip (31) is secured tightly in the upper portion (46) of the through-bore (42) by a close interference fit. A tight and effective seal is formed between the syringe tip (31) and adapter (10). Once the syringe (30) is locked in place, the clinician can inject the liquid (35) contained within the syringe chamber (39). The injected liquid (35) enters the wide-mouth lower portion (48) of the through-bore (42). The lower portion (48) provides a passageway for the liquid (35) to flow into the interior of the bottle (12) and dissolve the powdered drug (14) as described in further detail below.

As shown in FIG. 3, the tubular member (40) extends to a point slightly higher than the upper rim (38) of the adapter (10). Raising the tubular member (40) to this point ensures that it will make sufficient contact with the upper inside surface of the cap (26) when the cap is placed over the opening (24) of the bottle (12). The cap (26) is screwed onto the thread (28) of the neck portion (22) (FIG. 1A) as discussed above. The raised tubular member (40) presses against the inside surface of the cap (26) to form a seal. This helps prevent the powdered drug (14) from leaking as the bottle (12) is shipped, stored, and handled.

In practice, the medication bottle (12) is equipped with the adapter (10), as shown in FIG. 1, and covered with the cap (26) accordingly. The capped bottle (12) containing the adapter (10) is supplied to a clinician ready-for-use. It is preferred that the adapter (10) be installed in the bottle (12) during the packaging operation prior to shipping and handling of the bottle (12) by the end-user. With such a system, the clinician does not have to expend time and effort trying to properly position the adapter (10) in the bottle (12). Placing the cap (26) over the adapter (10) when the bottle (12) is being packaged provides an air-tight seal. The powdered drug (14) contained within the bottle (12) is kept clean and protected from dust, dirt, and other foreign debris. When a clinician is ready to use the medication bottle (12), he or she simply removes the cap (26) and inserts the tip (31) of the luer-lock syringe (30) into the tubular member (40). The luer-lock syringe (30) is filled with liquid (35) such as, for example, a liquid solvent for the powdered drug (14). The clinician guides the syringe tip (31) into the tubular member (40). The luer-lock syringe (30) is locked in place by twisting the syringe (30), normally in a clockwise direction, as discussed above. The adapter (10) is not threaded onto the bottle (12). Rather, the adapter (10) fits snugly into the neck portion (22) by a close interference fit, thereby providing a tight and secure seal. The clinician then depresses the plunger (37) on the syringe (30) and fills the bottle (12) with the liquid (35). The inter-locked syringe (30) and bottle (12) assembly can be rocked back and forth allowing the liquid (35) to fully dissolve the powdered drug (14). Furthermore, since the luer-lock syringe (30) is firmly secured to the bottle (12), the syringe/bottle assembly can be inverted completely.

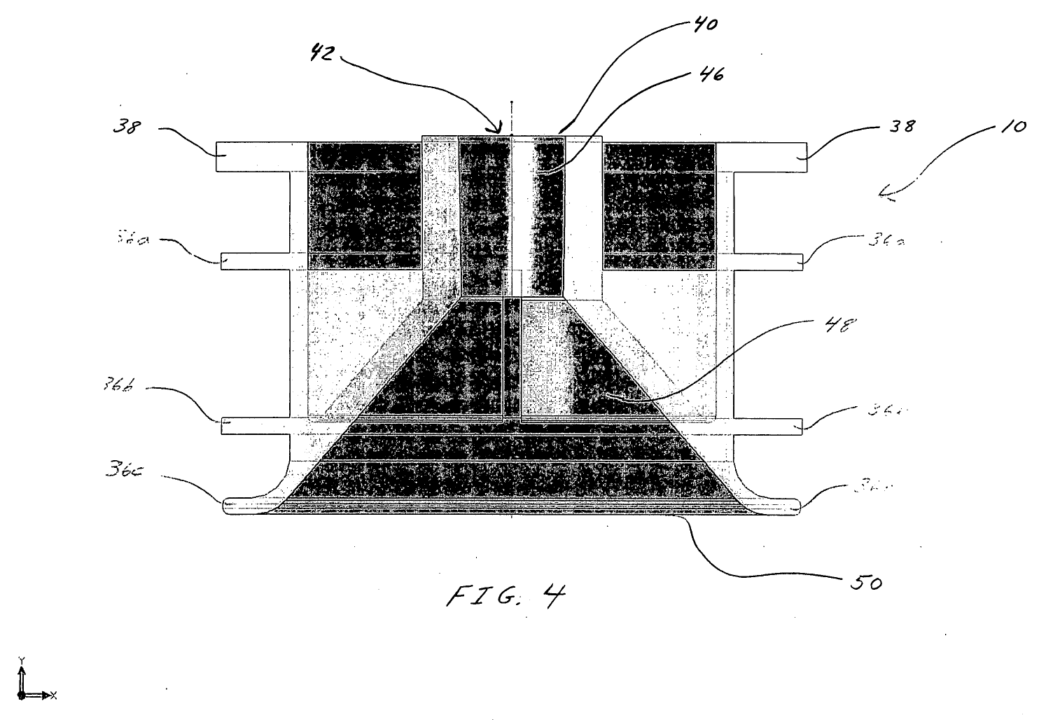

One advantageous feature of the adapter (10) of the present invention is the structure of the through-bore (42) extending through the tubular member (40). The wide-mouth lower portion (48) of the through-bore (42) (FIG. 3A) acts as a funnel, channeling fluid into the syringe (30) when the bottle (12) and attached syringe (30) are inverted. Once the powdered drug (14) has been fully dissolved, the clinician can withdraw the plunger (37) of the syringe (30). This action causes the drug solution to be pulled into the syringe chamber (39). Because of the above-described funneling effect, the clinician is better able to draw all of the drug solution into the syringe chamber (39). The funneling effect reduces the residual amount of drug solution, if any, remaining in the bottle (12). As shown in FIG. 4, it is preferred that the annular face (50) of the funnel-shaped lower portion (48) be substantially flush with the lower annular rib (36c) of the adapter (10). The funneling effect is improved when the face (50) is generally level with the lower annular rib (36c). The liquid (35) is channeled more effectively into the through-bore (42) and the residual amount of drug solution remaining in the bottle (12) is minimized.

If the clinician notices particulate in the drug solution during the drawing step, he or she can express the solution back into the bottle (12). Then, the clinician can repeat the step of rocking the inter-locked syringe (30) and bottle (12) assembly until the particulate fully dissolves. Finally, the clinician removes the syringe (30) from the bottle (12) by twisting the syringe (30) in a direction reverse to the direction used to lock the syringe (30). Normally, the syringe is twisted in a counter-clockwise direction to remove it from the adapter (10).

After the luer-lock syringe (30) has been withdrawn, the clinician can place a conventional needle, for example, a hypodermic needle or dental irrigation probe/needle, onto one end of the syringe (30). Then, the clinician can use the syringe (30), loaded with the drug solution, in a conventional manner.

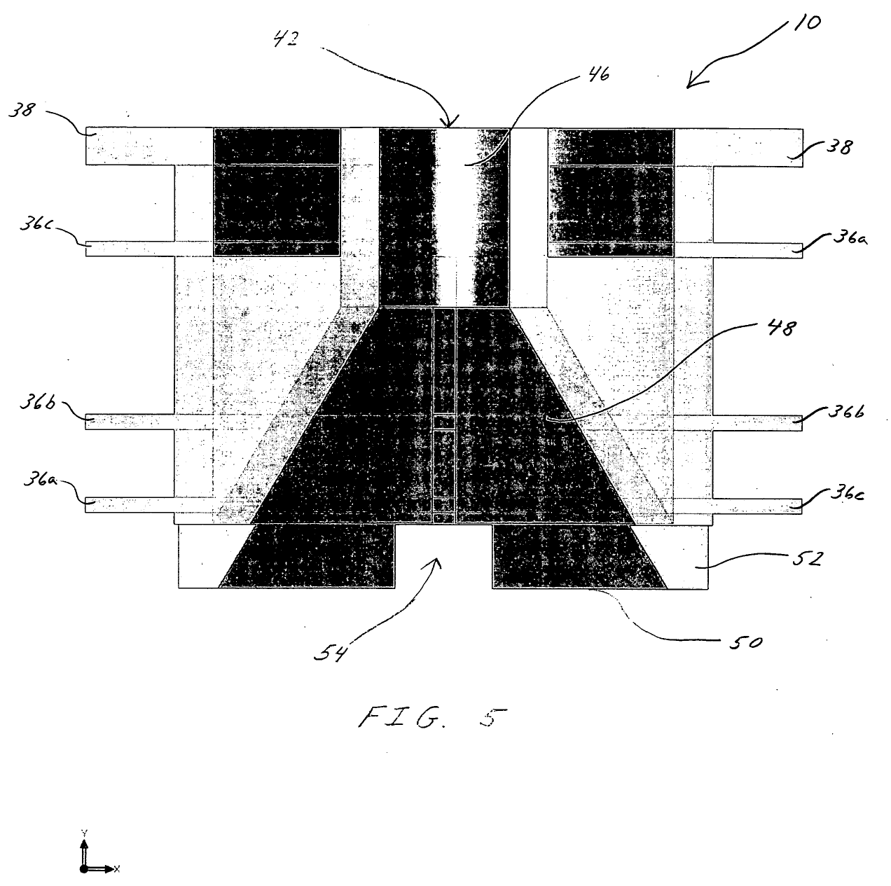

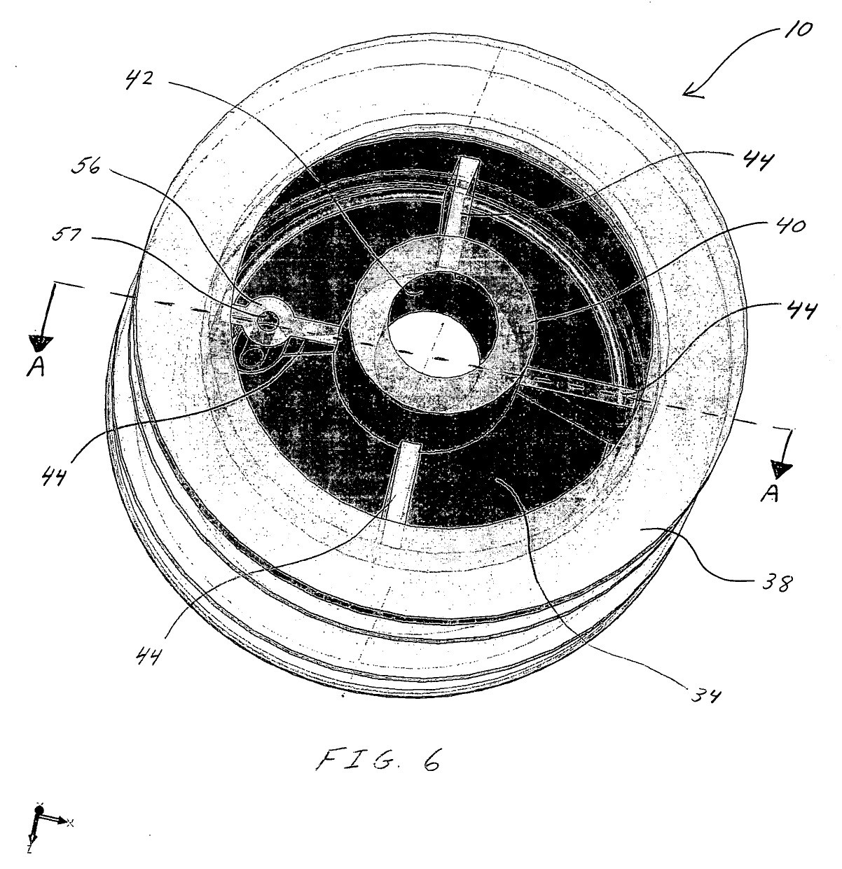

In another embodiment of this invention, as illustrated in FIG. 5, the adapter (10) includes a lower annular flange (52) extending from the wide-mouth lower portion (48) of the through-bore (40). The lower flange (52) makes up the lower face (50) of the through-bore (40). Thus, in this structure, the face (50) of the lower portion (48) is not substantially flush with the lower annular rib (36c). Rather, the face (50) rests below the lower rib (36c). The lower flange (52) also contains at least one drainage notch (54), which provides a pathway for the drug solution into and out of the through-bore (42). The lower flange (52) is shown having one drainage notch (54) for illustration purposes only and this should not be considered restrictive. Any suitable number of drainage notches (54) can be included in the lower flange (52). The drug solution is able to flow through the drainage notch (54) and into the through-bore (42) when the bottle (12) and attached adapter (10) are inverted. As discussed above, as the clinician draws the drug solution from the bottle (12), the solution flows into the through-bore (42) and is received in the syringe chamber (39). Because of this draining mechanism, the clinician is better able to completely draw the drug solution into the syringe (30). The residual amount, if any, of solution left in the bottle (12) is kept to a minimum.

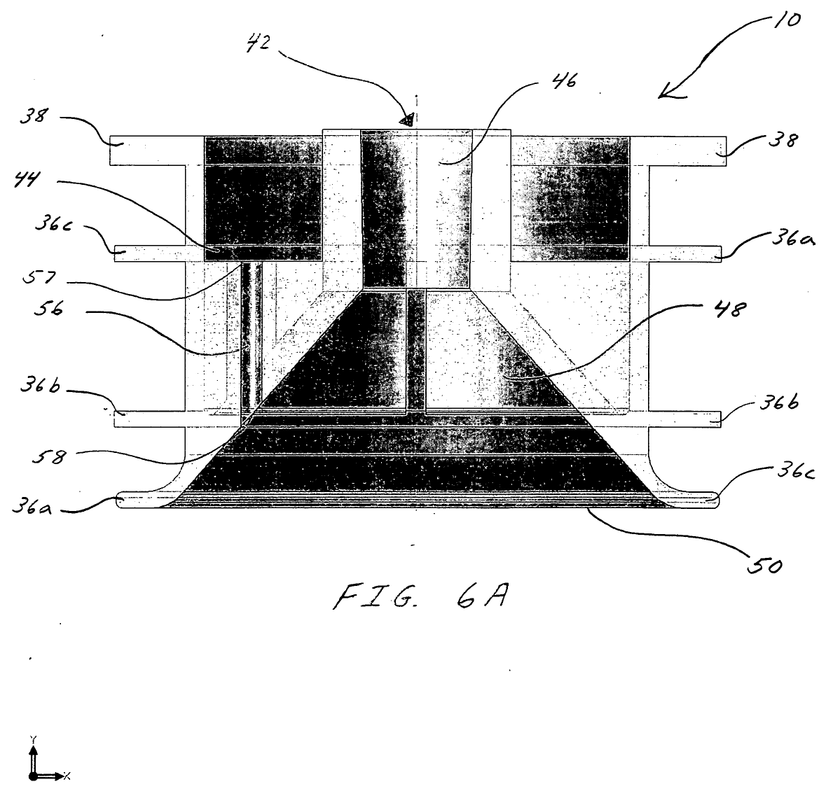

FIGS. 6 and 6A show yet another embodiment, wherein the adapter (10) includes a conduit (56) disposed in the upper recessed portion (34). The conduit (56) includes a top opening (57) extending through a support rib (44) and a bottom opening (58), which leads into the lower portion (48) of the through-bore (42). The conduit (56) extends in an axial direction and is parallel to the tubular member (40). Acting as an atmospheric vent, the conduit (56) relieves air pressure from the interior of the bottle (12). Pressurized air from the interior of the bottle (12) is vented to the atmosphere via the conduit (56). A pressure differential between the syringe (30) containing the liquid carrier (35) and the bottle (12) of powdered drug (14) can prevent the liquid (35) from initially flowing into the bottle (12). The vent (54) helps to relieve the air pressure inside of the bottle (12) so that the liquid (35) can flow more easily from the syringe (30) into the bottle (12). Also, by venting the air pressure within the bottle (12), the drug solution can be drawn more easily out of the bottle (12). When the inter-locked syringe (30) and bottle (12) assembly is inverted and the drug solution is drawn into the syringe (30), no air is introduced into the bottle (12). The inter-locked syringe (30) in the adapter (10) provides an air-tight seal. As a result, the drug solution is taken-up by the syringe (30), but the solution does not flow through conduit (56).

The adapter assembly (10) of the present invention has many advantageous features. For example, one advantage is that the adapter (10) can be sized to fit any medication bottle (12). The adapter (10) is not threaded onto the bottle (12). Rather, the adapter (10) snugly fits into the neck portion (22) of the bottle by a close interference fit, thereby providing a tight and effective seal. As discussed above, the adapter (10) is a unitary structure that can be molded from a polymeric material. The adapter (10) is less costly to manufacture than threaded adapters. Another advantage is that the adapter assembly (10) tightly secures and seals the luer-lock syringe (30) in place. As a result, the liquid (35) can be dispensed fully from the syringe (30) and into the medication bottle (12) with no spills or leaks. The clinician precisely knows the amount of liquid (35) and drug (14) being mixed. Also, the clinician can rock the bottle (12) and inter-locked syringe (30) assembly back and forth and even invert the assembly without worry of the solution leaking. The liquid (35) and drug (14) components can be mixed fully during this step with a reduced risk of contamination. Furthermore, once the liquid (35) and drug (14) components are mixed, the drug solution is ready-for-use. The clinician can withdraw the solution into the syringe (30), place a conventional needle thereon, and inject a patient with the solution. The clinician does not need to transfer the drug solution to another vial or container before administering it to a patient. This also helps reduce the risk of contamination and dosage errors.

The adapter (10) of this invention can be used for connecting a medication bottle (12) to a needleless syringe (30) in a wide variety of medical, dental, pharmaceutical, and other healthcare applications. The liquid (35) contained within the syringe (30) can be in any suitable form including, but not limited to, a carrier, diluent, reagent, or solvent. The drug (14) contained within the medication bottle (12) can take various forms including, but not limited to, liquids, solids, granules, powders, particles, tablets, capsules, spray-dried compounds, and lyophilized compounds.

In one preferred embodiment, the adapter (10) of this invention is used to make a drug solution for removing debris and bacteria from tooth surfaces during root canal operations. More particularly, the drug solution may comprise a disinfectant such as doxycycline hyclate; a detergent; an acid such as citric acid; and water. The drug solution is made from two parts. Part A of the product is a liquid carrier comprising water, polysorbate 80, and citric acid—this liquid part is stored in the luer-lock syringe (30). Part B is a doxycycline powder—the powder part is stored in the medication bottle (12) with attached adapter assembly (10).

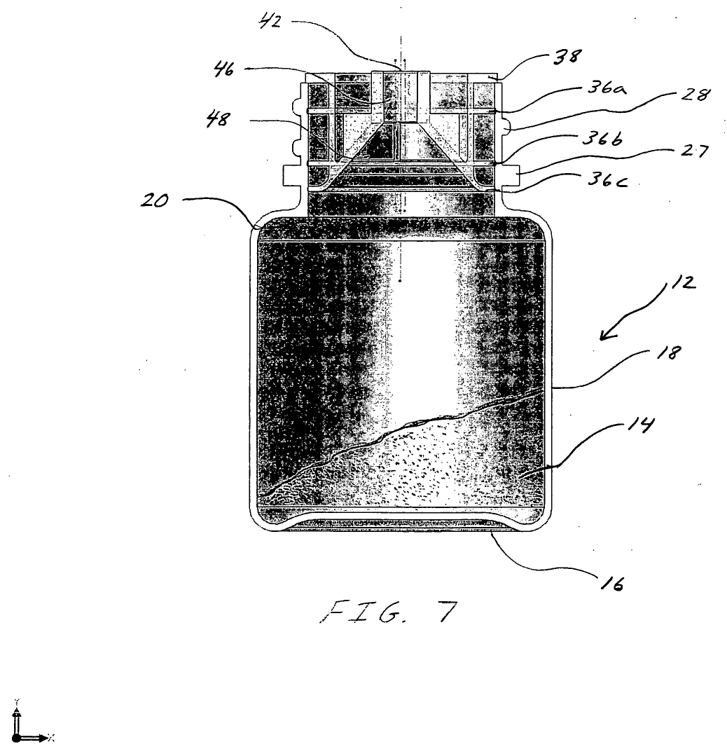

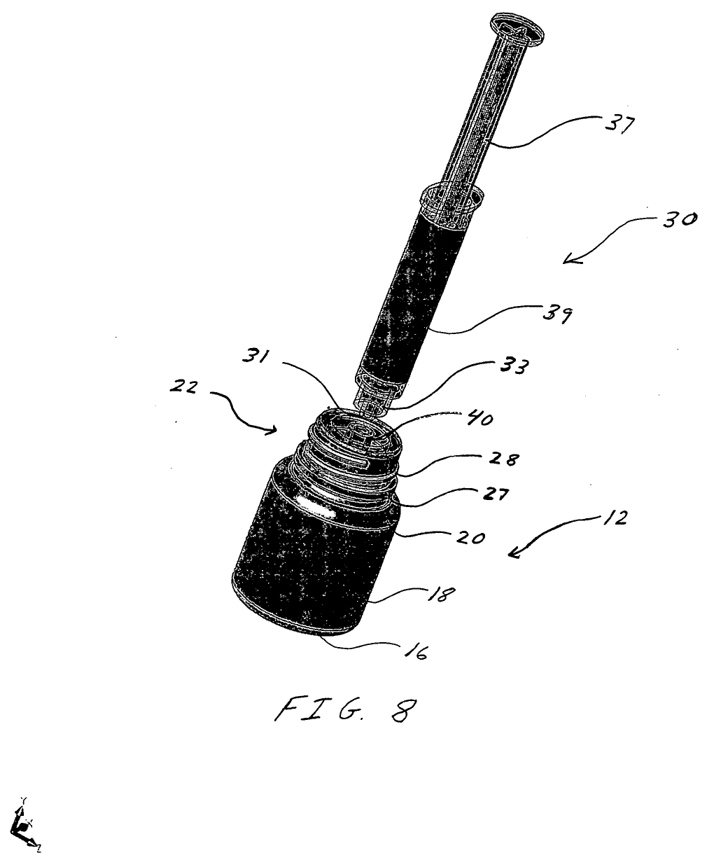

Referring to FIG. 7, the bottle (12) containing the powdered drug (14) and attached adapter assembly (10) is shown. In practice, a dentist inserts the luer-lock syringe (30) into the adapter (10) and locks the syringe (30) in place. FIG. 8 shows the luer-lock syringe tip (31) being inserted into the tubular member (40) of the adapter. Once the syringe (30) is inserted and locked in place, the dentist depresses the syringe plunger (37) to fill the bottle (12) with the liquid carrier (35). The inter-locked syringe (30) and bottle (12) assembly is gently rocked back and forth so that the liquid (35) can fully dissolve the powdered drug (14). Then, the dentist draws the drug solution into the syringe (30). The dentist is now ready to express the drug solution into the root canal of the tooth being treated. First, the dentist places an irrigation needle/probe onto the syringe (30). Then, the dentist slowly expresses some of the drug solution into the root canal of the tooth, allowing it to pool and soak into the root canal surfaces. During this step, the drug solution cleans and disinfects the canal. The remaining drug solution is used to flush loose necrotic tissue out of the canal. Such a root canal cleanser is available from Dentsply Tulsa (Tulsa, Okla.) under the brand name, BioPure™ MTAD™. Various compositions of the root canal cleanser and methods of using the cleanser are described in published United States patent applications, Publication Nos.: US 2003/0138383 and US 2003/0235804, the disclosures of which are hereby incorporated by reference. The foregoing example of dental treatment is but one example of how the adapter (10) of this invention can be used. It is recognized that the adapter (10) can be used in many other dental and medical applications.

Workers skilled in the art will appreciate that various modifications can be made to the embodiments and description herein without departing from the spirit and scope of the present invention. It is intended that all such modifications within the spirit and scope of the present invention be covered by the appended claims.

What is claimed is:

1. An adapter for connecting a luer-lock syringe containing a liquid to a bottle containing a drug in solid form, the bottle containing a neck portion with an opening, and the adapter comprising:
a main body member having a cylindrically-shaped structure with interior and exterior surfaces, the body member including an upper annular rim that defines an upper recessed portion;
a tubular member disposed within the recessed portion of the main body member, the tubular member having a through-bore extending in an axial direction through the body member, the tubular member including an upper portion for receiving the tip of the luer-lock syringe and a lower portion for providing a passageway for the liquid from the syringe to interior of the bottle; and
multiple annular fins attached to the exterior surface of the main body member, the fins including at least one upper fin and one lower fin, each of the fins extending outwardly for engaging interior surfaces of the bottle as the adapter is positioned in the neck of the bottle, the lower portion of the tubular member including a lower face that is substantially flush with the lower annular fin.

2. The adapter of claim 1, wherein the upper portion of the tubular member has a tapered cylindrical structure.

3. The adapter of claim 1, wherein the upper portion of the tubular member includes multiple support ribs extending outwardly therefrom.

4. The adapter of claim 1, wherein the lower portion of the tubular member has a conical structure.

5. The adapter of claim 1, wherein the upper portion of the tubular member has a tapered cylindrical structure and the lower portion of the tubular member has a conical structure, the inside diameter of the upper portion being less than the inside diameter of the lower portion.

6. The adapter of claim 1, wherein the upper annular rim of the main body member extends outwardly and overlays an upper rim of the bottle.

7. The adapter of claim 1, further comprising at least one intermediate annular fin, the intermediate fin being interposed between the upper annular fin and lower annular fin.

8. An adapter for connecting a luer-lock syringe containing a liquid to a bottle containing a drug in solid form, the bottle containing a neck portion with an opening, and the adapter comprising:
a main body member having a cylindrically-shaped structure with interior and exterior surfaces, the body member including an upper annular rim that defines an upper recessed portion;
a tubular member disposed within the recessed portion of the main body member, the tubular member having a through-bore extending in an axial direction through the body member, the tubular member including an upper portion for receiving the tip of the luer-lock syringe and a lower portion for providing a passageway for the liquid from the syringe to interior of the bottle; and
a lower annular flange extending from the lower portion of the tubular member, the flange including a drainage notch for providing a liquid passageway between the tubular member and interior of the bottle; and
multiple annular fins attached to the exterior surface of the main body member, the fins including at least one upper fin and one lower fin, each of the fins extending outwardly for engaging interior surfaces of the bottle as the adapter is positioned in the neck of the bottle, the lower portion of the tubular member including a lower face that is not flush with the lower annular fin.

9. An adapter for connecting a luer-lock syringe containing a liquid to a bottle containing a drug in solid form, the bottle containing a neck portion with an opening, and the adapter comprising:
a main body member having a cylindrically-shaped structure with interior and exterior surfaces, the body member including an upper annular rim that defines an upper recessed portion;
a tubular member disposed within the recessed portion of the main body member, the tubular member having a through-bore extending in an axial direction through the body member, the tubular member including an upper portion for receiving the tip of the luer-lock syringe and a lower portion for providing a passageway for the liquid from the syringe to interior of the bottle;
a conduit disposed within the recessed portion of the main body member, the conduit extending in an axial direction through the body member and parallel to the tubular member, the conduit having upper and lower openings for venting air pressure from the interior of the bottle; and
multiple annular fins attached to the exterior surface of the main body member, the fins including at least one upper fin and one lower fin, each of the fins extending outwardly for engaging interior surfaces of the bottle as the adapter is positioned in the neck of the bottle.

10. The adapter of claim 1, 2, or 3, wherein the liquid is a carrier for the drug, and the drug is in the form of a powder.

11. The adapter of claim 10, wherein the liquid comprises detergent, acid, and water, and the powdered drug comprises a disinfectant.

12. The adapter of claim 10, wherein the liquid contained in the luer-lock syringe is injected into the bottle containing the powdered drug to form a drug solution.

13. The adapter of claim 12, wherein the drug solution is used for dental applications.

14. The adapter of claim 12, wherein the drug solution is used for medical applications.
